(19) United States
(12) Patent Application Publication
(10) Pub. No.: US 2022/0032090 A1
(43) Pub. Date: Feb. 3, 2022

(54) ULTRASONIC THERMAL NASAL DECONGESTANT

(71) Applicant: Diego Gonzalez Joven, La Jolla, CA (US)

(72) Inventor: Diego Gonzalez Joven, La Jolla, CA (US)

(21) Appl. No.: 17/351,786

(22) Filed: Jun. 18, 2021

(30) Foreign Application Priority Data
Jul. 31, 2020 (MX) MX/E/2020/041997

(51) Int. Cl. A61N 7/00; A61F 7/00

(52) U.S. Cl. CPC A61N 7/00; A61F 7/007; A61F 2007/0071; A61F 2007/0006; A61N 2007/0004

(57) ABSTRACT

A thermal-vibratory device for the relief of nasal congestion including a flexible body that allows the device to be coupled to the average human face.

### FIELD OF INVENTION

This invention refers to an ultrasonic thermal nasal decongestant used in the medical and health field.

### SUMMARY OF INVENTION

This ultrasonic thermal nasal decongestant is a device that is placed over the nose, upper and inferior paranasal cavities, so that, with heat and ultrasonic micro-vibrations activate the operation of nasal cavities, causing the flow of mucus to activate by relieving allergy problems, inflammation, and other nasal discomforts.

### BACKGROUND OF THE INVENTION

In today's market, there are nasal decongestants, such as SniffRelief® https://sniffrelief.com/#features; due to the material and its structural, technical design, it is inefficient, unsustainable for the environment, expensive, and has some problems when used by the end-user.

The device of this invention has a technical, structural design of type “butterfly” configuration, in which the butterfly has a direct type of skin fastening. This is due to that the flexible flat surface of the butterfly and the material allows the device to adhere against the skin of the face, preventing different temperature drafts from entering and escaping the device, while state-of-the-art SniffRelief® is made of a fibrous material that causes the outside air go in and out between the skin and the SniffRelief®, which causes more energy consumption as well.

The material of the device of this invention has a greater benefit because it works like any vinyl film such as those that are glued to the glass, wherewith the pure vacuum that generates its flat surface, makes the material stick to the skin, creating a much larger seal, which is impossible to make with the fabric of the state-of-the-art SniffRelief®.

One of the most important technical characteristics of the device of this invention is that decongestion is carried out using ultrasonic vibration, supported by only the ultrasonic motors, with which, when mixed with centralized heat, the effects are much better.

Motors not only attack the area of the cavities but the nose, which the mask doesn't have.

On the other hand, for the state-of-the-art SniffRelief® to work better, you have to tighten it against the user's skin, which depends on slat holding. However, this device works without pressure and maximizes its functions without being held against the face. If the user wants to walk or has to be face down, it has a ribbon that attaches to it.

### BRIEF DESCRIPTION OF THE DRAWINGS

FIGS. 1a and 1b illustrate the ultrasonic thermal nasal decongestant 100 of this butterfly-shaped invention to relieve nasal congestion. This butterfly is controlled by a motherboard that houses rechargeable batteries and is controlled via power buttons and thermal-level controls. The resistors are printed on a flexible circuit.

FIGS. 2a and 2b illustrate a drawing of a user's skull showing the regions of the sinuses or paranasal cavities. These are the regions that the butterfly covers.

FIG. 3 illustrates the location of the rechargeable battery, control buttons, and motherboard.

FIG. 4 shows an ultrasonic motor.

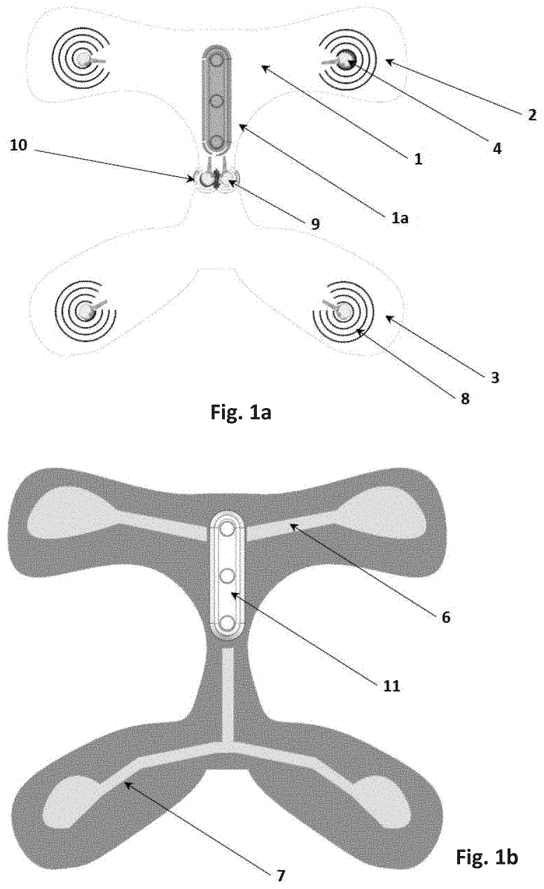

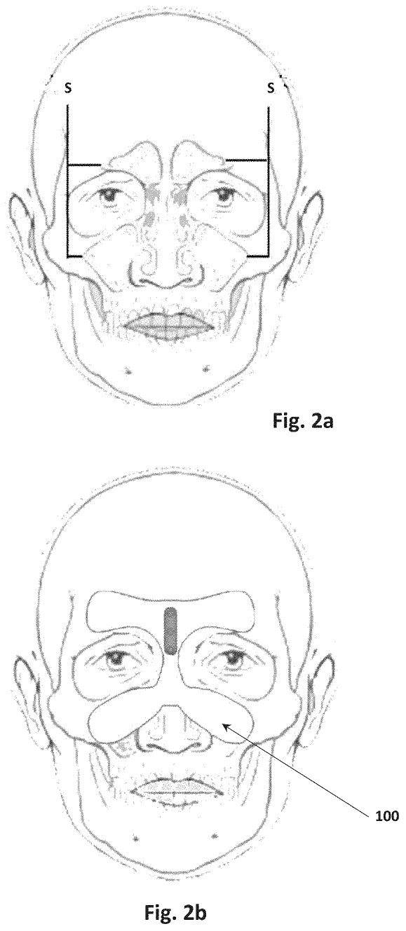

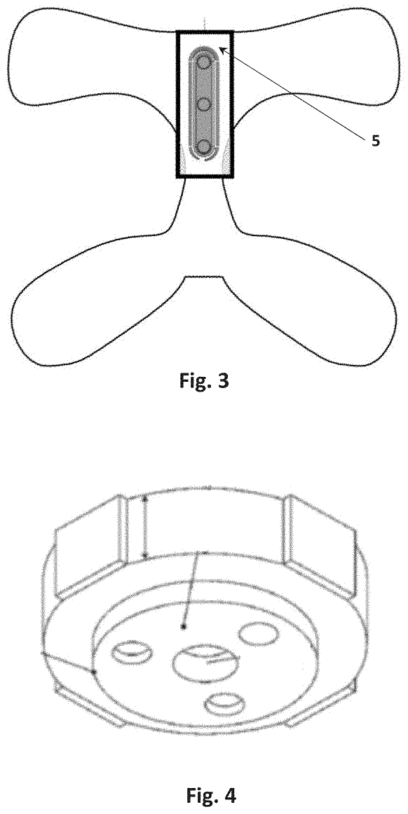

### DETAILED DESCRIPTION OF THE INVENTION

The ultrasonic thermal nasal decongestant 100 of this invention consists of a main body 1 of a butterfly-type configuration, which comprises upper wings 2 and lower wings 3 that come off from a central elongated core that is the support arch for the nose 1a, where the nose support arch 1a has a central motor 9 on each side and a central resistor 10 to generate heat, so that the nose support arch 1a is available on the septum of the user's nose and the upper wings 2 and lower wings 3 are arranged over 10 the user's sinuses S; at least one ultrasonic vibration motor 4 arranged in each one of the ends of the upper wings 2 and the lower wings 3, such motors can be 6 of the compact cylindrical ultrasonic type.

For automatic control of the device, a motherboard 5 is arranged within a housing in the central elongated core 1a from which higher resistances of 15 temperature 6 and lower temperature resistors 7 are provided throughout the butterfly-configuration main body 1. The system is housed on the motherboard control of the resistors and motors. This circuit contains a bluetooth connectivity module and a rechargeable battery.

The device of this invention may contain a resistor 8 around each engine 4 to generate heat. It also has circuits composed of a rechargeable battery, energy-efficient Bluetooth module, a housing 11 with control buttons such as on and off, + and − button to raise and decrease the temperature and vibrations, as well as a USB port for charging the battery, with LED bulbs indicating the level of battery charge.

The main body material 1 is neoprene which allows it to be washed with soap and water and facilitates temperature transmission.

The resistors 8, 10 are a copper layer printed on a flexible circuit which, when having an adjustable electric charge, generates a resistance that heats the upper-temperature resistors 6 and the lower-temperature resistors 7. These upper-temperature resistors 6 and the lower-temperature resistors 7 are above the sinuses and part of the nose bridge, which, when heated, allow areas where mucus is lodged to activate and release pressure and discharge mucus. Both motors and thermal resistors can be ignited independently, whether only the bridge of the nose or one of the four wings.

Ultrasonic motors, when activated, emit ultrasonic vibrations which stimulate the nerves from the region that is in contact and stimulates nasal flow. On each cavity, there is a motor, and on the bridge of the nose are two motors. The electronics of the device are integrated into the device completely.

The contact area of the device in the lower sinuses is much smaller by default than the main body; this main body does not have mechanical stress or retraction support against the skin. The main body is glued with all the stitches the vinyl neoprene sticks to the skin.

The use of components is much cheaper, less electronic and smaller, and the equal price is lower.

The figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. An ultrasonic thermal nasal decongestant, comprising:
a butterfly-like configuration main body with upper wings and lower wings emanating from a central elongated core so that the central core is laid over the septum of the user's nose and the upper wings and lower wings are arranged over the user's sinuses;
at least one ultrasonic vibration motor arranged at each end of the upper wings and lower wings;
a motherboard arranged in the central elongated core, from which high-temperature resistors are released, and lower temperature resistors are arranged over the butterfly-like configuration main body.

2. The ultrasonic thermal nasal decongestant in accordance with the claim 1, comprises a resistor around each engine to generate heat.

3. The ultrasonic thermal nasal decongestant in accordance with the claim 1, wherein the nose support arch has a central motor on each side and a central resistor to generate heat.

4. The ultrasonic thermal nasal decongestant in accordance with the claim 1, further comprising circuits including a rechargeable battery, low-power Bluetooth module, a housing with control buttons such as on/off and +/−buttons to raise and decrease the temperature and vibrations, as well as a USB port for charging the battery, with LED spotlights indicating the level of battery charge.
